(19) United States
(12) Patent Application Publication
(10) Pub. No.: US 2011/0017985 A1
(43) Pub. Date: Jan. 27, 2011

(54) ELECTRONIC DEVICE UTILIZING FLUORINATED CARBON NANOTUBES

(76) Inventors: Graciela Beatriz Blanchet, Boston, MA (US); Helen S.M. Lu, Wallingford, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/933,467

(22) PCT Filed: Apr. 1, 2009
(86) PCT No.: PCT/US09/39066

§ 371 Date: Sep. 20, 2010

Related U.S. Application Data
(60) Provisional application No. 61/041,605, filed on Apr. 2, 2008.

(51) Int. Cl. H01L 51/00; C07C 22/08; C07C 309/81; C07C 43/176; H01L 51/40

(52) U.S. Cl. 257/40; 570/128; 562/826; 568/660; 438/99; 977/746; 977/847; 977/936; 977/932; 257/E51.001; 257/E51.004

(57) ABSTRACT

The present invention is an electronic device and a process for making the electronic device in which the semiconductor component comprises at least one carbon nanotube functionalized with a fluorinated olefin. Functionalization with the fluorinated olefin renders the carbon nanotube semiconducting.

### FIELD OF THE INVENTION

The present invention is an electronic device and a process for making the electronic device in which the semiconductor component comprises at least one carbon nanotube functionalized with a fluorinated olefin.

### TECHNICAL BACKGROUND

Park et al (Physical Review B (2003) 68(4). 045429/1-045429/8) investigated stable adsorption geometries of fluorine atoms on a single-walled carbon nanotube using density-functional calculations.

Krusic et al (WO 2006/023921) describe carbon materials such as a fullerene molecule or a curved carbon nanostructure that are functionalized by addition chemistry performed on surface C—C double bond.

In printable electronics, there is a need for an electronic device and a process for making the electronic device in which the semiconductor component comprises at least one carbon nanotube functionalized with a fluorinated olefin.

### SUMMARY OF THE INVENTION

The present invention is an electronic device comprising a semiconductor component comprising at least one carbon nanotube that has been functionalized with a fluorinated olefin.

In addition, the invention is directed to an electronic device comprising: a) a semiconductor component comprising at least one carbon nanotube that has been functionalized with a fluorinated olefin; b) a source electrode; c) a drain electrode; d) a gate dielectric; and e) a gate electrode.

The invention is further directed to a composition comprising a carbon nanotube functionalized with a fluorinated olefin selected from the group consisting of perfluoro (5-methyl-3,6-dioxanon-1-ene), trifluoroethylene, 1-bromo-1-chlorodifluoroethylene, 1,1,2,3,3-pentafluoropropene, heptafluoro-1-butene, perfluorohexene, pentafluoroethyltrifluorovinyl ether, trifluoromethyl trifluorovinyl ether, heptafluoropropyltrifluorovinyl ether and mixtures thereof.

### BRIEF DESCRIPTION OF THE FIGURES

FIGS. 1A & B illustrate field effect transistors.

FIG. 2 illustrates TGA analysis of the sample of Example 1.

FIG. 3 illustrates a gate sweep of the sample of Example 1.

FIG. 4 illustrates a gate sweep of the sample of Example 2.

FIG. 5 illustrates a gate sweep of the sample of Example 3.

FIG. 6 illustrates an IV curve of the sample of Example 3.

FIG. 7 illustrates an on/off ratio of the sample of Example 4.

FIG. 8 illustrates a gate sweep of the sample of Example 5.

FIG. 9 illustrates an gate sweep of the sample of Example 6 run as a p-type transistor.

### DETAILED DESCRIPTION

The present invention is an electronic device and a process for making an electronic device comprising a semiconductor component comprising at least one carbon nanotube which has been functionalized by cycloaddition with a fluorinated olefin. The semiconductor component of the electronic device is a semiconducting material located between and in contact with the source and drain electrodes. Examples of the electronic device include transistors.

In an embodiment, carbon nanotubes are the semiconducting material in the semiconductor component of a field effect transistor. As produced, carbon nanotubes are a mixture of metallic conduction nanotubes and semiconducting nanotubes. Percolating arrays of mixtures of metallic and semiconducting nanotubes normally have their electrical conductivity dominated by the metallic-like tubes, which constitute about ⅔ of the carbon nanotube content, therefore, the array exhibits metallic-like conductivity. Such arrays would not be suitable for fabrication of the semiconductor component of the transistor because the array does not exhibit semiconductor activity. It has been found that functionalization of the carbon nanotubes by cycloaddition with a fluorinated olefin such as Perfluoro(4-Methyl-3,6-dioxaoct-7-ene)sulfonyl fluoride (PSEPVE, also known as 2-[1-[difluoro[(trifluoroethenyl)oxy]methyl]-1,2,2,2-tetrafluoroethoxy+]1,1,2,2-tetrafluoroethanesulfonyl fluoride. CAS [16090-14-5])) causes the nanotubes to exhibit primarily semiconducting behavior. Thus, percolating arrays on functionalized carbon nanotubes are mostly semiconducting and may be used to fabricate semiconductor components of transistors. It is further possible to construct a transistor in which the semiconductor is a single or several carbon nanotube. Functionalization of a plurality of carbon nanotubes by cycloaddition with fluorinated compounds would insure that individual nanotubes from a batch would be mostly semiconducting as well as functioning as the semiconductor component of the transistor.

Functionalization of carbon nanotubes by cycloaddition with fluorinated olefin convert carbon nanotubes to mostly semiconducting nanotubes. It is believed that the functionalization process converts C═C (carbon carbon double bond) sp2 carbon centers into C—C (carbon carbon single bond) sp3 C—C centers, thereby converting metallic tubes into semiconducting tubes. In this invention, functionalization is achieved by addition chemistry performed on surface C—C double bonds of a carbon nanostructure. One suitable method for performing an addition reaction is a cycloaddition reaction such as that of fluoroalkenes with themselves and other alkenes to form fluorocyclobutane rings. This is referred to herein as a “2+2” cycloaddition. Alternatively, fluoroalkenes could react with dienes in a “4+2” cycloaddition. Another suitable method is the addition of fluorinated radicals to the C—C double bond. These types of processes are described by Hudlicky in Chemistry of Organic Fluorine Compounds, 2nd ed, Ellis Horwood Ltd., 1976 and by Rico-Lattes, I. et al, Journal of Fluorine Chemistry, 107 (2001), 355-361.

In one embodiment of this invention, such a functionalization process may be performed in a reaction brought about by heating a carbon nanostructure material with a compound described by the general Formula 1

CF₂═CR¹R² Formula 1

wherein R¹ and R² are independently H, F, Cl, Br, CN, a branched or straight chain alkyl, alkylether, alkoxy, alkoxyether, fluororo-alkyl, fluoroalkylether, fluoroalkoxy, fluoroalkoxyether, aryl, aryloxy, fluoro-aryl, or fluoroaryloxy group; optionally substituted with one or more H, Cl, Br, carbinol, carboxylic acid ester, carboxylic acid halide, sulfonyl fluoride, or carbonitrile.

The above reaction will produce a functionalized carbon nanomaterial comprising n carbon atoms wherein m functional branches described generally by the Formula 2

C(F₂)—C(−)(R¹)—R² Formula 2

are each covalently bonded to the carbon nanotube through formation of a 4-member ring and/or a 6-member ring with the unsaturated pi system of the carbon nanotube.

The bonds resulting from opening a C═C bond in both the nanotube and a compound of Formula I, the ensuing 2+2 cycloaddition, create the 4-member ring. Furthermore, the bonds resulting from opening a C═C bond in both the nanotube and a compound of Formula I, the ensuing 2+4 cycloaddition, create the 6-member ring. As the ring itself is not shown in Formula 2, its presence is indicated by the incomplete bonds of the —C(F₂) and C(−) residues shown therein.

The compounds described in Formula I may be readily available commercially, or prepared in the manner set forth in U.S. Pat. No. 3,282,875 and U.S. Pat. No. 3,641,104 which are incorporated herein by reference. Some examples of commercially available fluorinated olefins include: tetrafluoroethylene, trifluoroethylene, 1-bromo-1-chlorodifluoroethylene, 1,1,2,3,3-pentafluoropropene, heptafluoro-1-butene, perfluorohexene, pentafluoroethyltrifluorovinyl ether, trifluoromethyl trifluorovinyl ether, heptafluoropropyltrifluorovinyl ether, perfluoro(4-methyl-3,6-dioxaoct-7-ene)sulfonyl fluoride, Perfluoro (5-methyl-3,6-dioxanon-1-ene).

In order to produce the functionalized carbon nanotubes, the nanotubes are contacted with the fluorinated olefin to form a mixture. The mixture of fluorinated olefin and nanotubes is heated to around 150-250 C for 5 to 24 hours, preferably from 180-220 C for 10 to 24 hours. The mixture is then washed extensively with solvents and dried. Thermogravimetric analysis of the product formed from contacting the carbon nanotube with fluorinated olefin can be performed and shows weight loss in the temperature range between 200 and 400 C. The dried carbon nanotubes may then be dispersed in a solvent such as o-dichlorobenzene, toluene, chloroform among others.

In one embodiment of this invention, a mixture of carbon nanotube and perfluoro(4-Methyl-3,6-dioxaoct-7-ene)sulfonyl fluoride (PSEPVE, also known as 2-[1-[difluoro[(trifluoroethenyl)oxy]methyl]-1,2,2,2-tetrafluoroethoxy+]1,1,2,2-tetrafluoroethanesulfonyl fluoride, CAS [16090-14-5]) was heated at around 215 C for 18-24 hours. The mole ratio of PSEPVE to carbon nanotube's C═C unit was from 0.1 to 8, preferably from 0.3 to 8, and more preferably from 0.3 to 2.

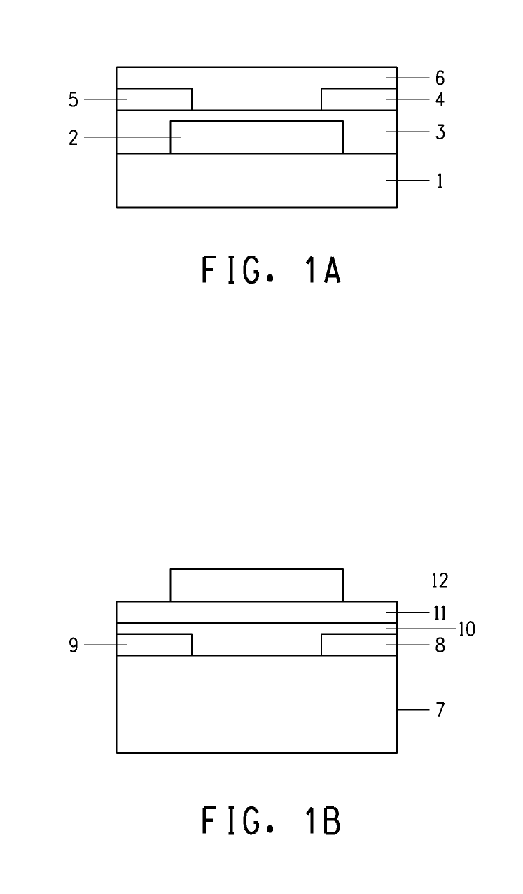

To fabricate a transistor of the present invention, the dispersion of functionalized carbon nanotubes in a solvent are deposited on a prefabricated partial transistor structure. The partial transistor structure contains other elements of the transistor which may be a gate electrode and a gate dielectric or a source and drain electrode. Standard transistors configurations are top gate and bottom gate. In the top gate configuration, the source and drain electrodes are deposited on the substrate with the semiconductor, gate dielectric and gate electrode deposited above them. In the bottom gate structure, the gate electrode is deposited on the substrate with the gate dielectric, semiconductor and source and drain electrode deposited above the gate electrode. The partial transistor structure is fabricated on a substrate in either the top gate or bottom gate configuration. A small space between the source and drain electrodes is referred to as the channel and is the location for the semiconductor component of the transistor. FIG. 1A illustrates a bottom gate configuration with the source and drain electrodes, 4 and 5 located on the gate dielectric, 3. The gate dielectric is located on at lease one of the sides of the gate electrode 2. At least one sides of the gate electrode is in contact with the substrate 1. The source and drain electrodes are electronic conductors and can be made by various methods such as evaporation, sputtering or by printing dispersions of metal particles in a solvent and drying the solvent. The semiconductor component 6 is made from a dispersion of functionalized carbon nanotubes. The dispersion of carbon nanotubes in a solvent is then deposited onto the source and drain electrodes on the gate dielectric for the bottom gate configuration. Spin coating, printing or ink jet printing may be used to deposit the semiconductor component of the dispersion of carbon nanotubes on the source and drain electrodes and then dried to allow evaporation of the solvent. The dried dispersion forms a percolating array of functionalized carbon nanotubes in the channel between and in contact with the source and drain electrodes. In top gate transistors as shown in FIG. 1b), the source and drain electrodes, 8 and 9 are deposited on the device substrate 7 and the semiconductor 10 comprising the carbon nanotubes is applied directly on top of the source and drain. A gate dielectric 11 which is an electrical insulator is then deposited on the semiconductor component. The gate dielectric may also be printed as a dispersion of metal oxide in a solvent. The gate electrode 12, a conductor, is then deposited on the gate dielectric. The gate electrode may also be a printed dispersion of metal particles in a solvent.

Alternatively, in a bottom gate configuration, the transistor may be fabricated such that the gate electrode is deposited directly on the substrate, or, as in a doped Si-wafer, the substrate is also the gate. The gate deposition is followed by the gate dielectric. The semiconductor component comprising the functionalized carbon nanotubes is then deposited on the gate dielectric and dried. Finally, the source and drain electrodes are deposited on the semiconductor component. Other arrangements of transistor components are also possible, but the semiconductor component is located between and in contact with the source and drain electrodes.

The semiconductor component comprising at least one carbon nanotube which has been functionalized by cycloaddition with a fluorinated olefin may also be used to fabricate other electronic devices such as diodes, solar cells, radio frequency ID tags, sensors, and any electronic device that uses a semiconductor material.

The present invention is also a composition comprising a carbon nanotube functionalized with a fluorinated olefin selected from the group consisting of perfluoro (5-methyl-3,6-dioxanon-1-ene), trifluoroethylene, 1-bromo-1-chlorodifluoroethylene;1,1,2,3,3-pentafluoropropene, heptafluoro-1-butene, perfluorohexene, pentafluoroethyltrifluorovinyl ether, trifluoromethyl trifluorovinyl ether, heptafluoropropyltrifluorovinyl ether and mixtures thereof. The carbon nanotubes may be functionalized by contacting the nanotube with the selected fluorinated olefin and heating the resulting mixture to about 215 C for several hours.

### EXAMPLES

### Example 1

### Synthesis of Fluorinated SWNTs

24.3 mg of purified Hipco carbon nanotubes (CNI, Incorporated. Austin Tex.) were heated with 0.5 mL PSEPVE (Perfluoro(4-Methyl-3,6-dioxaoct-7-ene)sulfonyl fluoride, also known as 2-[1-[difluoro[(trifluoroethenyl)oxy]methyl]-1,2,2,2-tetrafluoroethoxy+]1,1,2,2-tetrafluoroethanesulfonyl fluoride. CAS [16090-14-5], DuPont, Wilmington Del.) for 215° C. for

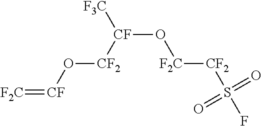

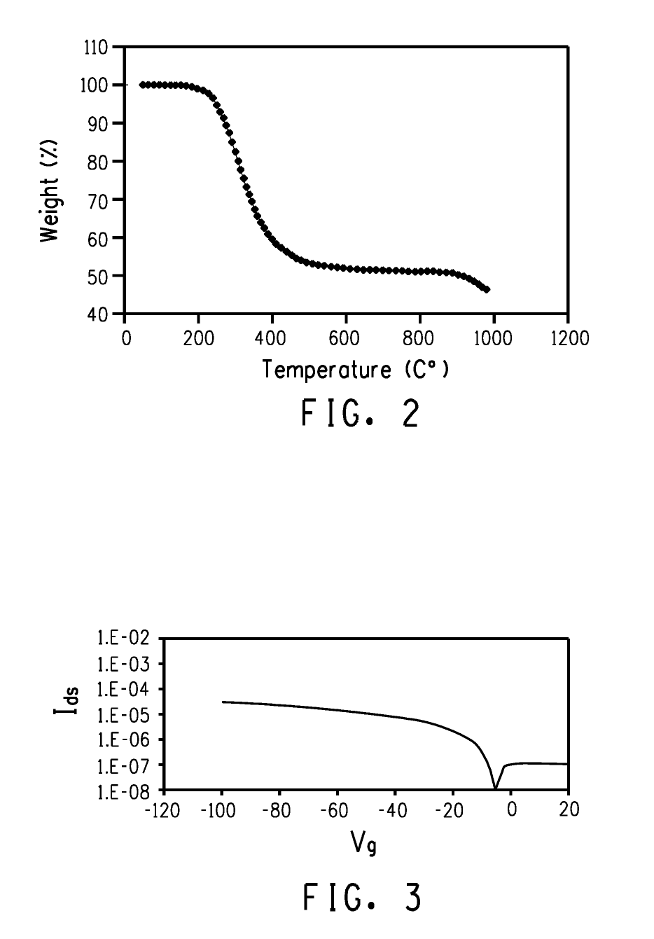

24 h. The chemical structure of PSEPVE is shown above. The products were washed extensively with acetone and Vetrel-XF. The product was dried at 175° C. for 2 hour. The final mass was 32.5 mg. Thermogravimetric analysis (TGA) shows approximately 45% wt loss. The TGA of the fluorinated tubes is shown in FIG. 2.

The functionalized carbon nanotubes obtained from the procedure above were then dispersed in o-dichlorobenzene (ODCB) at a concentration of 300 mg/L. The mixture was place in a 20 mL sonicated in the horn sonicator for 10 minutes at 22% of full power (750 watts). The dispersions were found to be stable even after two weeks.

The dispersions were then coated onto a clean Si/SiO₂ wafer with pre-patterned with a source and a drain electrodes. The oxide layer was 1500 A in thickness. The wafers were rinsed with acetone, followed by isopropyl alcohol and was finally rinsed with ultrapure water followed by drying with a nitrogen gun. The wafers were then plasma cleaned for 1 minute in an Argon atmosphere prior to the spinning of the carbon nanotube dispersion. Then the spin coating is done at 100 rpm for 60 sec. The wafer was then placed on the hotplate at 65° C. for around 30 minutes. The wafer was then placed in Nitrogen glove box for electrical characterization. The electrical properties were measured using a standard Agilent unit 4155C, California City, Calif. The gate sweep of a device with W/L=200/20 in example 1 is shown below as FIG. 3. The source drain voltage was set to −2 Volts and the gate voltage was swept from 10 V to −100V as shown. The saturated mobility was calculated to be 0.6 cm²/Vsec and the on/off ratio was 1.79×10³.

### Example 2

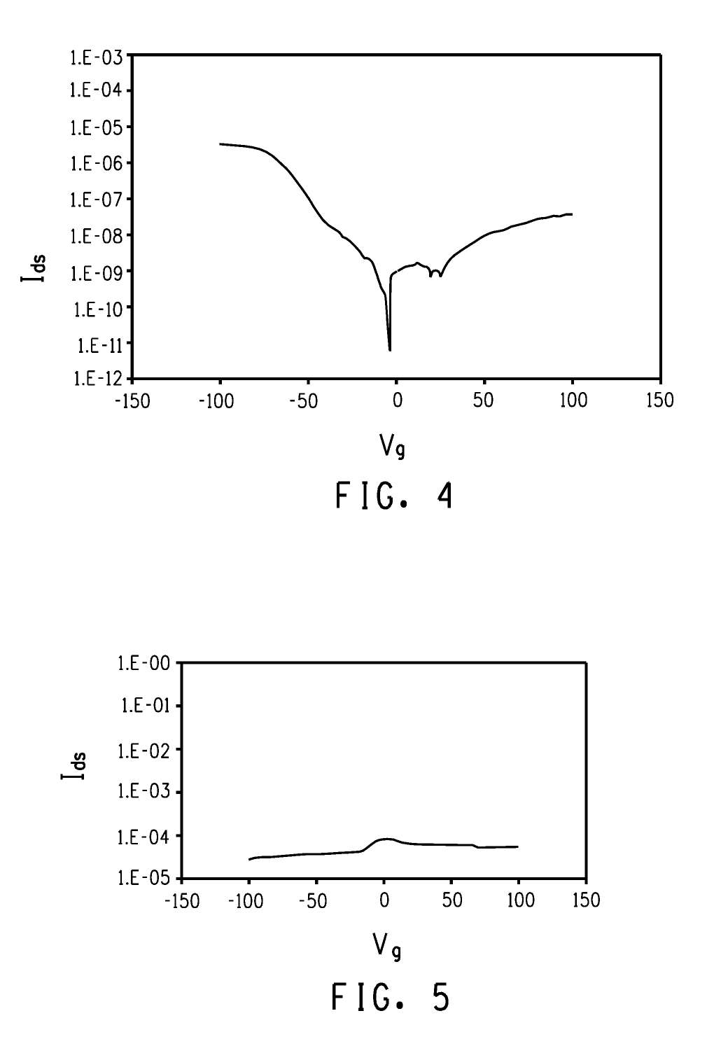

A device was prepared as in example 1 but the source/drain voltage was set to 0.1 Volts and the gate voltage was swept from −100 to 100 Volts as shown in FIG. 4. The saturated mobility and on/off ratio were 8.8 cm²/V sec and 4.7 10⁵ respectively.

As shown in the examples above fluorinated nanotubes are semiconducting, ambipolar and have on/off ratios>10³. while the non fluorinated counterpart (example 3 below) has metallic behavior and an on/off ratio of 3.

### Example 3

A control sample using Hipco-non fluorinated material was used. The commercial Hipco carbon nanotubes were dispersed in o-dichlorobenzene (ODCB) at a concentration of 300 mg/L. The mixture was place in a 20 mL sonicated in the horn sonicator for 10 minutes at 22% of full power and spun onto clean Si/SiO wafers with pre-patterned Au sources-drains as indicated above. The gate sweep is shown below in FIG. 5 for Vsd: −5 V and Vg 100 to −100 V. As expected from a percolating array with metallic character the lon/loff of 2.54 is very low.

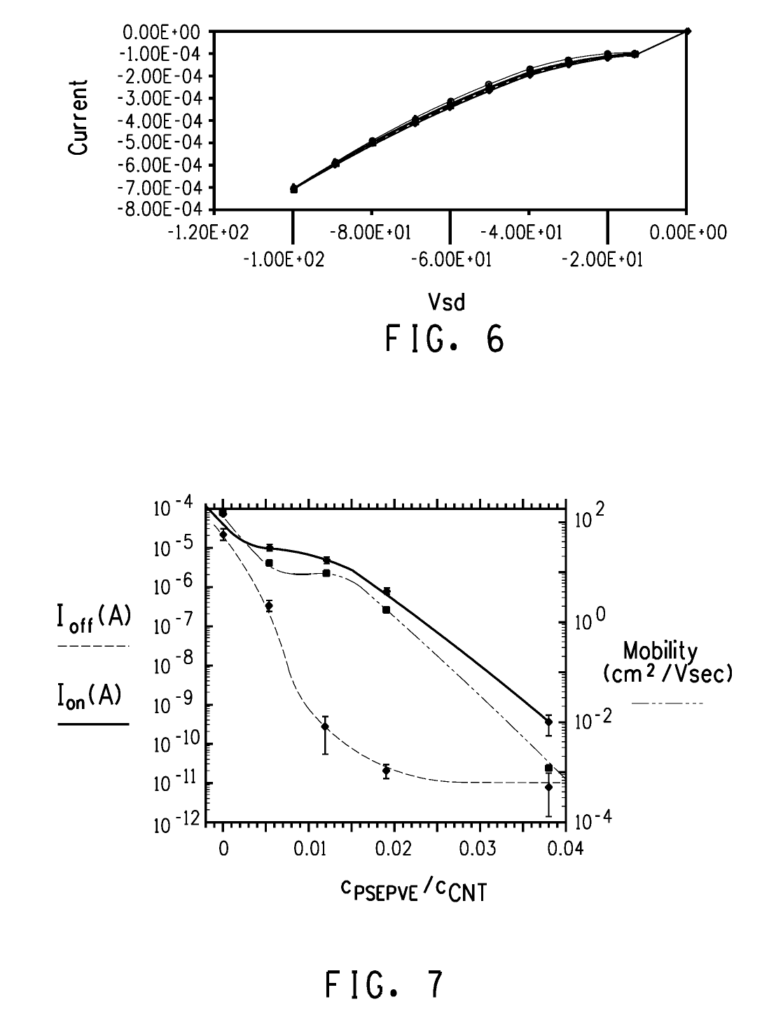

The IV curves of these tubes further corroborate the metallic behavior. As shown in the IV curves in FIG. 6, the current voltage characteristic does not change by changing the gate voltage. The nominal saturated mobility is 2.98×10⁴.

### Example 4

In Example 4, 24.3 mg of purified Hipco carbon nanotubes (CNI, Incorporated. Austin Tex.) were heated with 0.1, 0.3, 0.5 and 2 mL PSEPVE (Perfluoro(4-Methyl-3,6-dioxaoct-7-ene)sulfonyl fluoride, also known as 2-[1-[difluoro[(trifluoroethenyl)oxy]methyl]-1,2,2,2-tetrafluoroethoxy+]1,1,2,2-tetrafluoroethanesulfonyl fluoride. CAS [16090-14-5]) respectively at 215° C. for

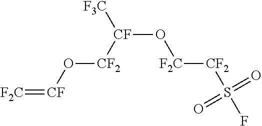

24 h. The chemical structure of PSEPVE is shown above. The products were washed extensively with acetone and Vetrel-XF. The products were dried at 175° C. for 2 hour.

The functionalized carbon nanotubes obtained from each of the procedures above were then dispersed in o-dichlorobenzene (ODCB) at a concentration of 300 mg/L. Each mixture was place in a 20 mL sonicated in the horn sonicator for 10 minutes at 22% of full power (750 watts). The dispersions were found to be stable even after two weeks. The dispersions were then coated onto a clean Si/SiO2 wafer with pre-patterned with sources and drains. The oxide layer was 1500 A in thickness. The wafers were rinsed with acetone, followed by isopropyl alcohol and was finally rinsed with ultrapure water followed by drying with a nitrogen gun. The wafers were then plasma cleaned for 1 minute in an Argon atmosphere prior to the spinning of the carbon nanotube dispersion. Then the spin coating is done at 100 rpm for 60 sec. The wafer was then placed on the hotplate at 65° C. for around 30 minutes. The wafer was then placed in Nitrogen glove box for electrical characterization. The electrical properties were measured using a standard Agilent unit 4155C, California City, Calif. The gate sweep of devices with W/L=200/20 was run and the off current, mobility and on/off ratio tabulated The effect of a systematic [cycloaddition reaction on the mobility and off current (l_{off}) of a percolating array of FSWNT is illustrated for FSWNT-PSEPVE in FIG. 7. The dramatic reduction of the l_{off} with increasing reactant concentration (c_{PSEPVE}/c_{CNT}, the ratio of the moles of reactant that successfully reacted to the moles of SWNT C═C units) is key to this work. c_{PSEPVE}/c_{CNT}, is calculated from the weight gained from the reaction divided by the molecular weight of PSEPVE divided by the mole of the carbon nanotube's C═C unit. In this example, 0.1 mL of PSEPVE led to c_{PSEPVE}/c_{CNT} of 0.005, 0.3 mL of PSEPVE led to c_{PSEPVE}/c_{CNT} of 0.012, 0.5 mL of PSEPVE led to c_{PSEPVE}/c_{CNT} 0.019 and 2 mL of PSEPVE led to c_{PSEPVE}/c_{CNT} of 0.034.

Devices fabricated from a percolating array of pristine HiPco tubes have high mobilities but also high l_{off}, which indicates that conduction pathways are dominated by metallic tubes. Increasing PSEPVE functionalization led to a dramatic decrease in l_{off}, caused by a reduction in the number of metallic percolating pathways. For one embodiment the concentration ratio range is 0.007<c_{PSEPVE}/c_{CNT}<0.02. In another embodiment, the range is 0.005<c_{PSEPVE}/c_{CNT}<0.035. For 0.007<c_{PSEPVE}/c_{CNT}<0.02, high mobility is preserved, but l_{off} was reduced by almost 5 orders of magnitude as compared to pristine SWNTs. For another embodiment, the concentration ratio is 0.007<c_{PSEPVE}/c_{CNT}<0.02. At higher reactant concentrations, the mobility dropped precipitously, which suggests that the electronic properties of the M and SC-SWNTs have changed considerably. The field effect mobilities deduced from the linear regime are 10 cm²/V·sec with on/off ratios in excess of 10⁵.

The highest mobilities with on/off ratios on the order of 10⁵ are obtained in the 0.3-0.5 ml PSEPVE addition level. Further increases the PSEPVE addition level rapidly degrades the mobility. The drain voltage was set to −0.1 Volts and the gate voltage was swept from 10 V to −100V as shown.

### Example 5

Example 5 illustrates the functionalization of single wall carbon nanotubes (SWNT) with Perfluoro (5-methyl-3,6-dioxanon-1-ene) (CAS [1644-11-7], Synquest Laboratory, Inc. Alachua, Fla.) whose structure is shown below:

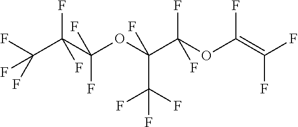

24 mg of the commercially purified HiPCO SWNTs were dried at 250° C. at a pressure of <1 mbar, for overnight and was then transferred in to a 10 mL stainless steel tube reactor. 0.5 mL of the perfluoro(5-methyl-3,6-dioxaanon-1-ene) (Mol wt=432.06) was added to the tube. The stainless steel tube reactor was closed under nitrogen, chilled in dry ice for 30 minutes, and then evacuated to remove the N2. The stainless tube reactor was heated with agitation at 215° C. for 24 hours. The products were washed extensively with acetone and Vetrel-XF to remove the residual fluorinated olefin and were filtered through a 0.2 micron PTFE membrane. The recovered functionalized carbon nanotubes were dried at 175° C. under vacuum for 2 hours. The functionalized SWNTs were then dispersed in ODCB at a concentration of 300 mg/L and was horn sonicated for 10 minutes.

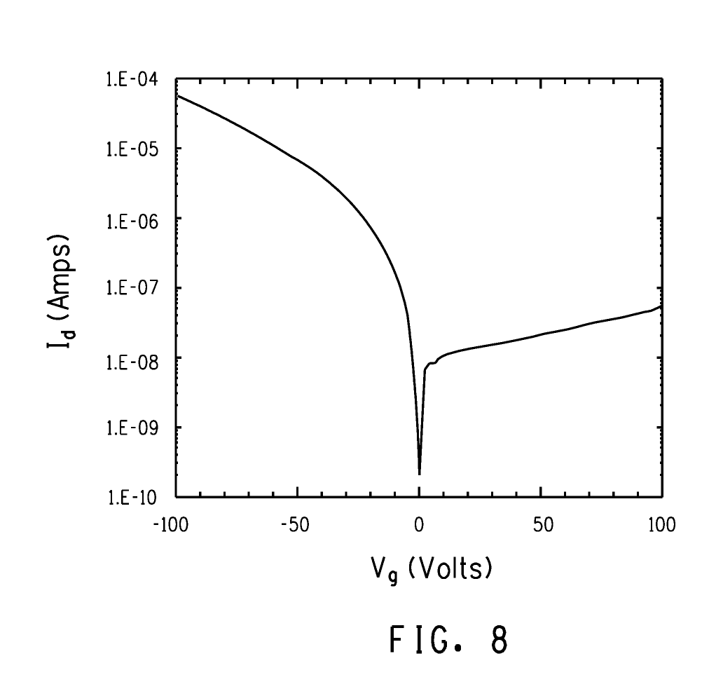

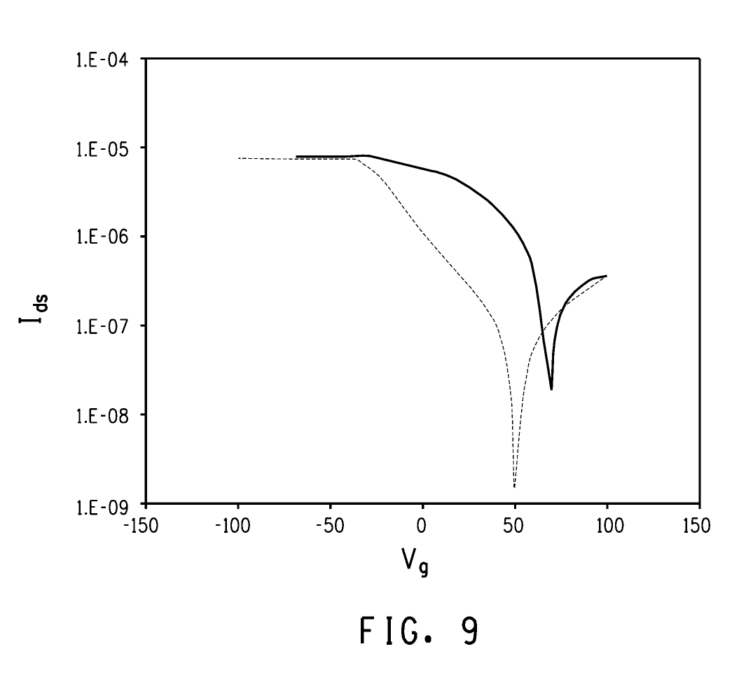

The dispersions were then coated onto a clean Si/SiO2 wafer with pre-patterned with sources and drains. The oxide layer was 1500 A in thickness. The wafers were rinsed with acetone, followed by isopropyl alcohol and was finally rinsed with ultrapure water followed by drying with a nitrogen gun. The wafers were then plasma cleaned for 1 minute in an Argon atmosphere prior to the spinning of the carbon nanotube dispersion. Then the spin coating is done at 100 rpm for 60 sec. The wafer was then placed on the hotplate at 65° C. for around 30 minutes. The wafer was then placed in Nitrogen glove box for electrical characterization. The electrical properties were measured using a standard Agilent unit 4155C, California City, Calif. The gate sweep of devices with W/L=200/20 was run and the mobility of 110 cm2/Vsec and on/off ratio of 3×10⁵ calculated shown in FIG. 8.

### Example 6

Single-walled carbon nanotubes (SWNTs) were functionalized with Tetrafluoroethylene (TFE) (CF2=CF2) 24 mg of the commercially purified HiPCO SWNTs were dried at 250° C. at a pressure of <1 mbar, for overnight. Then the tubes were transferred to glass reactor vessel. The reactor vessel was purged by nitrogen gas to remove the residual oxygen gases and moisture. Tetrafluoroethylene was then introduced to the reaction vessel and the pressure was maintained at 20 psi. The reaction vessel was heated at 215° C. overnight with constant shaking. The products were washed extensively with acetone and Vertrel XF and were filtered through a 0.2 micron PTFE membrane. The recovered functionalized carbon nanotubes were dried at 175 C under vacuum for 2 hours. The functionalized SWNTs were then dispersed in ODCB at a concentration of 300 mg/L and was horn sonicated for 10 minutes.

The dispersions were then coated onto a clean Si/SiO₂ wafer with pre-patterned with sources and drains. The oxide layer was 1500 A in thickness. The wafers were rinsed with acetone, followed by isopropyl alcohol and was finally rinsed with ultrapure water followed by drying with a nitrogen gun. The wafers were then plasma cleaned for 1 minute in an Argon atmosphere prior to the spinning of the carbon nanotube dispersion. Then the spin coating is done at 100 rpm for 60 sec. The wafer was then placed on the hotplate at 65° C. for around 30 minutes. The wafer was then placed in Nitrogen glove box for electrical characterization. The electrical properties were measured using a standard Agilent unit 4155C, California City, Calif. The gate sweep of devices with W/L=200/20 shown in FIG. 9, was run and the mobility of 10.8 cm2/Vsec and on/off ratio of 5.22×10³ calculated.

What is claimed is:

1. An electronic device comprising a semiconductor component wherein the semiconductor component comprises at least one carbon nanotube functionalized with a fluorinated olefin.

2. An electronic device of claim 1 wherein the at least one carbon nanotube is a percolating array of carbon nanotubes.

3. The electronic device of claim 1 further comprising:
a) a source electrode;
b) a drain electrode;
c) a gate dielectric; and
d) a gate electrode.

4. The electronic device of claim 1 wherein the fluorinated olefin is selected from the group consisting of perfluoro(4-methyl-3,6-dioxaoct-7-ene)sulfonyl fluoride, perfluoro (5-methyl-3,6-dioxanon-1-ene), tetrafluoroethylene, trifluoroethylene, 1-bromo-1-chlorodifluoroethylene, 1,1,2,3,3-pentafluoropropene, heptafluoro-1-butene, perfluorohexene, pentafluoroethyltrifluorovinyl ether, trifluoromethyl trifluorovinyl ether, heptafluoropropyltrifluorovinyl ether and mixtures thereof.

5. The electronic device of claim 4 wherein the fluorinated olefin is perfluoro(4-methyl-3,6-dioxaoct-7-ene)sulfonyl fluoride and the concentration ratio is cₚₛₑₚᵥₑ/c_{cnt} is between 0.005 and 0.035.

6. The electronic device of claim 4 wherein the fluorinated olefin is perfluoro(4-methyl-3,6-dioxaoct-7-ene)sulfonyl fluoride and the concentration ratio is cₚₛₑₚᵥₑ/c_{cnt} is between 0.007 and 0.02.

7. The electronic device of claim 1 wherein the electronic device is a transistor.

8. A process comprising:
a) providing a substrate comprising source and drain electrodes;
b) depositing at least one carbon nanotube functionalized with a fluorinated olefin on the substrate.

9. The process of claim 8 wherein the at least one carbon nanotube is a percolating array.

10. A process comprising:
a) providing a substrate;
b) depositing at least one carbon nanotube functionalized with a fluorinated olefin on the substrate; and
c) depositing source and drain electrodes on the array of carbon nanotubes.

11. The process of claim 10 wherein the at least one carbon nanotube is a percolating array.

12. The process of claims 8 wherein the fluorinated olefin is selected from the group consisting of perfluoro(4-methyl-3,6-dioxaoct-7-ene)sulfonyl fluoride, perfluoro (5-methyl-3,6-dioxanon-1-ene), tetrafluoroethylene, trifluoroethylene, 1-bromo-1-chlorodifluoroethylene, 1,1,2,3,3-pentafluoropropene, heptafluoro-1-butene, perfluorohexene, pentafluoroethyltrifluorovinyl ether, trifluoromethyl trifluorovinyl ether, heptafluoropropyltrifluorovinyl ether and mixtures thereof.

13. The process of claims 10 wherein the fluorinated olefin is selected from the group consisting of perfluoro(4-methyl-3,6-dioxaoct-7-ene)sulfonyl fluoride, perfluoro (5-methyl-3,6-dioxanon-1-ene), tetrafluoroethylene, trifluoroethylene, 1-bromo-1-chlorodifluoroethylene, 1,1,2,3,3-pentafluoropropene, heptafluoro-1-butene, perfluorohexene, pentafluoroethyltrifluorovinyl ether, trifluoromethyl trifluorovinyl ether, heptafluoropropyltrifluorovinyl ether and mixtures thereof.

14. A composition comprising a carbon nanotube functionalized with a fluorinated olefin selected from the group consisting of perfluoro (5-methyl-3,6-dioxanon-1-ene), trifluoroethylene, 1-bromo-1-chlorodifluoroethylene, 1,1,2,3,3-pentafluoropropene, heptafluoro-1-butene, perfluorohexene, pentafluoroethyltrifluorovinyl ether, trifluoromethyl trifluorovinyl ether, heptafluoropropyltrifluorovinyl ether and mixtures thereof.
